(19) United States
(12) Patent Application Publication
(10) Pub. No.: US 2008/0274180 A1
(43) Pub. Date: Nov. 6, 2008

(54) Extended Release Pharmaceutical Composition of Metformin and a Process for Producing It

(75) Inventors: Shripad Rhushikesh Jathar, Maharashtra (IN); Rajesh Prabhamal Sirwani, Maharashtra (IN)

Correspondence Address: MERCHANT & GOULD PC, P.O. BOX 2903, MINNEAPOLIS, MN, 55402-0903, US

(73) Assignee: Nicholas Piramal India Limited, Mumbai (IN)

(21) Appl. No.: 12/065,334

(22) PCT Filed: Aug. 22, 2006
(86) PCT No.: PCT/IB06/52892

§ 371 Date: Jul. 7, 2008

(30) Foreign Application Priority Data
Aug. 30, 2005 (IN) 1035/MUM/2005

(51) Int. Cl. A61K 9/22; A61P 3/10

(52) U.S. Cl. 424/468

(57) ABSTRACT

A pharmaceutical composition in the form of tablets constitutes an orally administered, controlled drug delivery system that will provide increased retention time of the device in the stomach over conventional dosage forms and release metformin or its pharmaceutically acceptable salt in a controllable manner, and further that is easy and inexpensive to manufacture.

### FIELD OF INVENTION

The present invention relates to an extended release drug delivery composition of pharmaceutically active compound. The present invention particularly relates to an extended release drug delivery composition of a freely water-soluble pharmaceutically active agent.

### BACKGROUND OF THE INVENTION

Non-insulin dependent diabetes mellitus (NIDDM) is a progressive metabolic disorder with diverse pathologic manifestations and is often associated with lipid metabolism and glycometabolic disorders. The long-term effects of diabetes result from its vascular complications; the microvascular complications of retinopathy, neuropathy and nephropathy and the macrovascular complications of cardiovascular, cerebrovascular and peripheral vascular diseases. Initially, diet and exercise is the mainstay of treatment of type II diabetes. However, these are followed by administration of oral hypoglycemic agents. Current drugs used for managing type II diabetes and its precursor syndromes such as insulin resistance include classes of compounds, such as, among others, biguanides, thiazolidinediones and sulfonylureas.

For many disease states the ideal dosage regimen is that by which an acceptable therapeutic concentration of drug at the site of action is attained immediately and is then maintained constant for the duration of the treatment. Provided dose size and frequency of administration are correct, therapeutic ‘steady-state’ plasma concentrations of a drug can be achieved promptly and maintained by the repetitive administration of conventional peroral dosage forms. However, there are a number of potential limitations associated with conventional peroral dosage forms. These limitations have led pharmaceutical scientists to consider presenting therapeutically active molecules in ‘extended-release’ preparations.

Historically, oral drug administration has been the predominant route for drug delivery. An ideal oral drug delivery system should steadily deliver a measurable and reproducible amount of drug to the target site over a prolonged period. Controlled-release (CR) delivery systems provide a uniform concentration/amount of the drug at the absorption site and thus, after absorption, allow maintenance of plasma concentrations within a therapeutic range, which minimizes side effects and also reduces the frequency of administration. CR products are formulations that release active drug compounds into the body gradually and predictably over a 12- to 24-hour period and that can be taken once or twice a day. Typically, these products provide numerous benefits compared with immediate-release drugs, including greater effectiveness in the treatment of chronic conditions, reduced side effects, greater convenience, and higher levels of patient compliance due to a simplified dosing schedule. Because of the above advantages, such systems form the major segment of the drug delivery market.

Over the years many drug delivery systems have been developed with the aim of eliminating the peaks and troughs in plasma drug concentration seen after the administration of a conventional delivery system. A variety of terms have been used to describe these systems: delayed release, repeat action, prolonged release, sustained release, extended release, controlled release and modified release. It is interesting to note that the USP considers that the terms controlled release, prolonged release, sustained release and extended-release are interchangeable.

The basic concepts of controlled drug delivery are well known to those skilled in the art. Considerable efforts have been made in the last decades to develop new pharmaceutically viable and therapeutically effective controlled drug delivery systems. Attention has been focused particularly on orally administered controlled drug delivery systems because of the ease of administration via the oral route as well as the ease and economy of manufacture of oral dosage forms such as tablets and capsules. A number of different oral controlled drug delivery systems based on different release mechanisms have been developed. These oral controlled drug delivery systems are based on different modes of operation such as for example, dissolution controlled systems, diffusion controlled systems, ion-exchange resins, osmotically controlled systems, erodible matrix systems, swelling controlled systems, and the like.

An orally administered controlled drug delivery system encounters a wide range of highly variable conditions, such as pH, peristalsis, and ionic and enzymatic composition of the gastrointestinal fluids as it passes down the gastrointestinal tract. Ideally, an oral controlled drug delivery system will deliver the drug at a constant and reproducible rate in spite of the varying conditions. Considerable efforts have therefore been made to design oral controlled drug delivery systems that overcome these drawbacks and deliver the drug at a constant rate as it passes down the gastrointestinal tract.

It is well known to those skilled in the art that a drug may not be absorbed uniformly over the length of the gastrointestinal tract, and that drug absorption from the colon is usually erratic and inefficient. Also, certain drugs are absorbed only from the stomach or the upper parts of the small intestine. Furthermore, an important factor, which may adversely affect the performance of an oral controlled drug delivery system, is that the dosage form may be rapidly transported from more absorptive upper regions of the intestine to lower regions where the drug is less well absorbed. Therefore, in instances where the drug is not absorbed uniformly over the gastrointestinal tract, the rate of drug absorption may not be constant in spite of the drug delivery system delivering the drug at a constant rate into the gastrointestinal fluids. More particularly, in instances where a drug has a clear cut “absorption window,” i.e., the drug is absorbed only from specific regions of the stomach or upper parts of the small intestine, it may not be completely absorbed when administered in the form of a typical oral controlled drug delivery system. It is apparent that for a drug having such an “absorption window,” an effective oral controlled drug delivery system should be designed not only to deliver the drug at a controlled rate, but also to retain the drug in the upper parts of the gastrointestinal tract for a long period of time.

Metformin hydrochloride is freely soluble in water (>300 mg/ml at 25.degree. C.). It is absorbed extensively from the upper proximal region of the gastrointestinal tract and has poor absorption from the distal region. The absolute bioavailability of a 500 mg metformin hydrochloride tablet given under fasting conditions is approximately 50-60%. It shows a lack of dose proportionality with increasing doses due to decreased absorption indicating a saturable absorption process or permeability/transit time limited absorption. It has a plasma elimination half-life of about 3 hours that makes it a suitable candidate for extended release formulations.

Extended-release tablets have been described in the prior art and many methods have been used to provide extended-release pharmaceutical dosage forms in order to maintain therapeutic serum levels of medicaments and to minimize the effects of missed doses of drugs caused by a lack of patient compliance.

Osmotic drug delivery systems, makes use of the osmotic pressure as the driving force for delivery of the drugs. The osmotic drug delivery system comprises of an osmotic core that consists of a drug with or without an osmagent, which is coated with a semipermeable membrane and a delivery orifice is created with a mechanical or laser drill. U.S. Pat. Nos. 3,845,770; 3,916,899; 4,034,758; 4,077,407; 4,612,008; 4,783,337; 5,071,607; 5,082,668; 6,099,859 are few representative prior art references that makes use of the osmotic drug delivery system. A major disadvantage of the above-described system is that mechanical or laser drilling is capital intensive. Also, the size of the hole is critical so also is the integrity and consistency of the coating essential. If the coating process is not well controlled there is a risk of film defects, which could result in dose dumping and the film droplets must be induced to coalesce into a film with consistent properties.

Multiporous Oral Drug Absorption System as described in for example U.S. Pat. No. 5,505,962 is surrounded by a non-disintegrating, timed-release coating, which after coming in contact with gastrointestinal fluid is transformed into semipermeable membrane through which the drug diffuses in a rate-limiting manner. A disadvantage of this is that the coating, since it requires a pore forming agent, cannot provide a uniform coating and therefore the release rate may not be uniform from one tablet to another.

U.S. Pat. Nos. 4,915,952; 5,328,942; 5,451,409; 5,783,212; 5,945,125; 6,090,411; 6,120,803; 6,210,710; 6,217,903; PCT publication nos. WO 96/26718; WO 97/18814 describes the use of polymer matrices to achieve controlled release which is achieved either by limiting the rate by which the surrounding gastric fluid can diffuse through the matrix and reach the drug, dissolve the drug and diffuse out again with the dissolved drug, or by using a matrix that slowly erodes thereby continuously exposing fresh drug to the surrounding fluid. A disadvantage of matrices that erode more readily however is that they cause a high initial burst of drug release and a lower degree of control over the drug release rate over the initial course of the drug release.

U.S. Pat. Nos. 5,007,790; 5,582,837; 5,972,389; 6,340,475; 6,495,162; 6,723,340 WO 98/55107 describes achieving gastric retention by swelling, wherein the dosage form when ingested, swells to a size that is large enough so as to prevent from passing through the stomach into the intestine. Though, gastric retention is achieved efficiently, the disadvantage of swellable system is the time required to swell and therefore it could result in a lag time before the blood level concentrations are seen. Further, the swelling can cause blockade of the pyloric sphincter and lead to other complications.

U.S. Pat. No. 6,261,601 describes a pharmaceutical composition, which provides a combination of spatial and temporal control of drug delivery by making use of controlled gas powered technology. The disadvantage of the composition is that, it would not be suitable for a highly water-soluble drug.

PCT publications WO01/10417, WO00/06129 and poster presented at 141^{st} British Pharmaceutical Conference by Dave et. al. describes a pharmaceutical composition wherein an additional acid source is used along with the gas generating agent. Stability of a composition having an acid-base couple is of concern thereby reducing the shelf life.

PCT publication WO2005/060942 describes a pharmaceutical composition claiming a gastric retention system, but has no details about the release profile as well as about the plasma profile.

Several controlled release metformin formulations are now available in the market, but these existing formulations come along with the above-mentioned disadvantages. Accordingly, none of the oral controlled drug delivery systems described is completely satisfactory. Therefore, there remains a need for an improved pharmaceutical composition for delivering metformin from a pharmaceutical composition at a sustained rate avoiding the disadvantages of the presently known compositions.

As can be observed in the above-referenced patents and publications, compositions have been described that provide for prolonged delivery of an active agent and retention in the gastric environment. However, there remains a continuing need for improved systems for delivering an active agent to the gastric environment over a prolonged period of time and in a reliable, controllable and reproducible manner. In particular, there is a need for sustained delivery devices that are to remain in the stomach. Such devices should exhibit a combination of flexibility and rigidity so as not to be expelled from the stomach through the pyloric sphincter, and deliver active agent in a reproducible, controlled manner, over a prolonged period of time.

### OBJECTS OF THE INVENTION

Therefore, the basic object of the present invention is to provide an extended release pharmaceutical composition for delivering a freely water soluble pharmaceutically active agent at a controlled rate avoiding the said disadvantages of the compositions known in the art.

Another object of the present invention is to provide an extended release pharmaceutical composition that is capable of delivering steadily a measurable and reproducible amount of a freely water soluble pharmaceutically active agent to the target site over a prolonged period.

Yet another object of the present invention is to provide an extended release composition that is capable of releasing a freely water soluble pharmaceutically active compound into the body gradually and predictably over a 12 to 24 hour period, and therefore may be administered once or twice in a day.

Another object of the present invention is to provide a drug delivery system that is capable of providing a uniform concentration of a freely water soluble pharmaceutically active agent at the absorption site.

Another object of the present invention is to provide a drug delivery system that is capable of providing a uniform concentration of a freely soluble pharmaceutically active agent having a narrow absorption window at the absorption site thereby allowing maintenance of plasma concentrations within a therapeutic range, minimizing the side effects and reducing the frequency of administration.

Another object of the present invention is to provide an extended release drug delivery system that exhibits greater effectiveness in the treatment of chronic conditions and ensures high levels of patient compliance.

Yet another object of the present invention is to provide an extended release drug delivery system that can deliver a freely water soluble active agent at a controlled rate, and that can simultaneously retain the said active agent in the upper parts of the gastrointestinal tract for a long duration.

A particular object of the present invention is to provide an extended release drug delivery system that exhibits a combination of flexibility and rigidity so as not to be expelled from the stomach through the pyloric sphincter and therefore deliver a freely water soluble pharmaceutically active agent in a reproducible and controlled manner over a prolonged period of time.

Still another object of the present invention is to provide a pharmaceutical composition having a mean time to maximum plasma concentration (Tₘₐₓ) of metformin at from 2.0 to 4.0 after the administration of dose.

Still another object of the present invention is to provide a pharmaceutical composition having a mean maximum plasma concentration (Cₘₐₓ) of metformin from about 450 ng/ml to about 650 ng/ml after administration of 500 mg of metfromin.

Yet another object of the present invention is to provide a pharmaceutical composition in the form of tablets, which constitute an orally administered, controlled drug delivery system that provides increased retention time of the device in the stomach over conventional dosage forms and releases a pharmaceutically active agent or its pharmaceutically acceptable salt in a reliably controllable manner, and that is further easy and inexpensive to manufacture.

Yet another object of the present invention is to provide a pharmaceutical composition that makes effective use of two or more hydrophilic or hydrophobic polymers so as to provide a desired release profile of a highly water soluble drug having a short absorption window.

### SUMMARY OF THE INVENTION

Accordingly, the present invention provides pharmaceutical composition comprising metformin or a pharmaceutically acceptable salt thereof as an active ingredient, a gas-generating agent, a hydrophilic polymer as a release retardant, one more hydrophilic or hydrophobic polymer to provide stability to the system and an additional hydrophilic polymer or gum as a release modifier.

In another embodiment, the present invention provides a process for preparation of said pharmaceutical composition, said process comprising the steps of:
i. Dissolving the binder in isopropyl alcohol followed by granulating metformin or its pharmaceutically acceptable salt and the hydrophilic and/or hydrophobic polymer and the additional hydrophilic polymer or gum.
ii. Passing the resultant wet mass obtained in step (I) above through a sieve and drying the resultant wet granules in a drier;
iii. Resizing the resultant dried granules obtained in step (ii) above and further mixing the same with a gas generating component and other excipients including lubricant, glidant, binder and/or filler.
iv. Compressing the resultant lubricated blend to a tablet.

### BRIEF DESCRIPTION OF THE DRAWINGS

FIG. 1 is a graph depicting plasma profile of the test composition and a standard composition available on the market.

### DETAILED DESCRIPTION OF THE INVENTION

The present invention is directed to a pharmaceutical composition in the form of tablets, which constitutes an orally administered, extended release drug delivery system for the treatment of non-insulin dependent diabetes mellitus in humans that will provide increased retention time of the device in the stomach over conventional dosage forms and release metformin or its pharmaceutically acceptable salt in a reliably controllable manner, and further that is easy and inexpensive to manufacture. The said pharmaceutical composition comprises metformin or a pharmaceutically acceptable salt thereof preferably hydrochloride salt of metformin as an active ingredient, a gas-generating agent, a hydrophilic or hydrophobic polymer as a release retardant, disintegrant, one more hydrophilic polymer to provide stability to the system and additionally a hydrophilic polymer or a gum as release modifier and optionally other pharmaceutical excipients.

Examples of the gas generating agent that can be used in the present invention include carbonates such as sodium carbonate or potassium carbonate; bicarbonates such as sodium bicarbonate or potassium bicarbonate. Preferably, the gas generating agent is selected from bicarbonates such as magnesium carbonate, sodium bicarbonate or potassium bicarbonate. The most preferred gas generating agent is sodium bicarbonate.

Examples of hydrophilic or hydrophobic polymer as a release retardant that can be used in the present invention include hydrophilic polymers such as hydroxyl ethyl cellulose, polyvinylpyrrolidone in combination with poly(vinyl alcohol), hydroxypropylcellulose, hydroxymethylcellulose, hydroxypropylmethylcellulose, gelatin, polyacrylic acid (carbopol), polyethyleneoxide and the like. Polymer blends are also suitable; hydrophobic polymers such as Eudragit®, Compritol®, polypropylene oxide, polyethylene, polypropylene, polycarbonate, polystyrene, polysulfone, polyphenylene oxide and polytetramethylene ether. Preferably, the hydrophilic or hydrophobic polymer as a release retardant polymer is hydroxypropylmethylcellulose, polyvinylpyrrolidone, carbopol, polyethyleneoxide, Eudragit®, Compritol®, polypropylene oxide, polyethylene or polyphenylene oxide. The most preferred hydrophilic or hydrophobic polymer as a release retardant polymer is hydroxypropylmethylcellulose, carbopol, polyethyleneoxide, hydroxyethylcellulose, Eudragit®, Compritol®.

Examples of disintegrants that can be used in the present invention include crospovidone, croscarmellose sodium, sodium starch glycolate, low-substituted hydroxypropyl cellulose. Sodium starch glycolate is the preferred disintegrant.

Examples of additionally one more hydrophilic polymer to provide stability to the system that can be used in the present invention include hydrophilic polymers such as sodium carboxymethylcellulose, hydroxypropylmethylcellulose, hydroxyethylcellulose. Preferably, the additional hydrophilic polymer is selected from sodium carboxymethylcellulose or hydroxypropylmethylcellulose.

Examples of the additional hydrophilic polymer or a gum as a release modifier that can be used in the present invention include sodium carboxymethylcellulose, guar gum, gum arabic, locust bean gum, xanthan gum preferably sodium carboxymethylcellulose and guar gum.

Optionally, the tablet may contain other pharmaceutically acceptable excipients such as lubricants, binders, fillers and glidant or anti adherent. Examples of commonly known lubricants include stearic acid, magnesium stearate, glyceryl behenate, stearyl behenate, talc, mineral oil (in polyethylene glycol), sodium stearyl fumarate and the like. Magnesium stearate is the most preferred lubricant. Examples of binders include water-soluble polymer, such as modified starch, gelatin, polyvinylalcohol (PVA), povidone (PVP). Povidone is the most preferred binder. Examples of fillers include lactose, microcrystalline cellulose, etc., the latter being preferred. An example of a glidant is silicon dioxide (Aerosil®). The above binders, lubricants, fillers, glidants, and any other-excipient that may be present can further be found in the relevant literature, for example in the Handbook of Pharmaceutical Excipients.

According to an embodiment of the invention, the relative amounts of the ingredients are as follows. The proportion of metformin or a pharmaceutically acceptable salt thereof may vary between about 55 and about 70% w/w, preferably about 60 to about 65% w/w. Proportion of the gas generating component may vary between about 5 and about 15% w/w, preferably about 7 to about 10% w/w. The proportion of the hydrophilic and/or hydrophobic polymer and the optional hydrophilic polymer or gum may vary between about 5 and about 50% w/w, preferably about 7.5 to about 35% w/w.

According to another embodiment of the invention there is provided a pharmaceutical composition having a mean time to maximum plasma concentration (Tₘₐₓ) of metformin at from 2.0 to 4.0 hours after the administration of dose.

According to still another object of the present invention there is provided a pharmaceutical composition having a mean maximum plasma concentration (Cₘₐₓ) of metformin from about 450 ng/ml to about 650 ng/ml after administration of 500 mg of metfromin.

Yet another embodiment of the present invention provides a metformin extended release tablet exhibiting a release profile such that after two hours, between about 10% to about 45% of the metformin or its pharmaceutically acceptable salt is released; after about four hours, between about 40% to about 65% of the metformin or its pharmaceutically acceptable salt is released; after about six hours, between about 60% to about 80% of the metformin or its pharmaceutically acceptable salt is released; after about eight hours, between 75% to about 95% of the metformin or its pharmaceutically acceptable salt is released; after about twelve hours not less than 95% of the metformin or its pharmaceutically acceptable salt is released.

Still another embodiment of the invention provides a metformin extended release tablet which when immersed in 0.1 N hydrochloric acid, floats on the surface within about 0.5 minute to about 5 minutes and continues to float for a period between about 6 hours to about 10 hours.

According to yet another embodiment of the present invention, there is provided a process for preparation of the pharmaceutical composition, which comprises:
i. Dissolving the binder in isopropyl alcohol followed by granulating metformin or its pharmaceutically acceptable salt and the hydrophilic and/or hydrophobic polymer and the additional hydrophilic polymer or gum.
ii. The resultant wet mass obtained in step (i) above is passed through a sieve and the resultant wet granules dried in a drier.
iii. The resultant dried granules obtained in step (ii) above are resized and further mixed with the gas generating component and other excipients that include lubricant, glidant, binder and/or filler.
iv. The resultant lubricated blend is then compressed to a tablet so as to provide about 500 mg of metformin or its pharmaceutically acceptable salt.

### EXAMPLES

The present invention is illustrated by, but is by no means limited to the following examples:

### Example 1

This example illustrates the present invention in the form of controlled release tablets of metformin hydrochloride wherein a combination of a hydrophobic polymer (ethocel), hydrophilic polymer (hydroxypropylmethylcellulose) and a third hydrophilic polymer (sodium CMC) is used to prepare the tablets. The pharmaceutical composition of this example is given in Table 1.

TABLE 1

| Ingredients | Quantity in mg per tablet |
|---|---|
| Metformin hydrochloride | 500 |
| Microcrystalline cellulose (MCC) | 59 |
| Povidone (PVP K-30) | 15 |
| Ethocel 100 cp | 65 |
| Sodium bicarbonate | 70 |
| Hydroxypropylmethylcellulose K-100 M | 65 |
| Sodium carboxymethylcellulose | 15 |
| (Cekol ® 10000A) | |
| Sodium Starch Glycolate | 4 |
| Aerosil ® 200 | 5 |
| Magnesium stearate | 2 |

i. Binder solution was prepared by dissolving povidone in isopropyl alcohol.
ii. Metformin hydrochloride that was sieved through 80 mesh sieve, microcrystalline cellulose, ethocel and hydropropylmethylcellulose were mixed properly and granulated with the binder solution of step (i).
iii. The wet mass obtained in step (ii) was passed through 8 mesh sieve and dried in a drier.
iv. The dried granules obtained in step (iii) were resized through 18 mesh sieve and blended with sodium bicarbonate, sodium carboxymethylcellulose, sodium starch glycolate, Aerosil® and magnesium stearate.
v. The resultant lubricated blend obtained in step (iv) is compressed to a tablet so as to provide about 500 mg of metformin hydrochloride.

### Example 2

This example illustrates the present invention in the form of controlled release tablets of metformin hydrochloride wherein a combination of a hydrophilic polymers hydroxypropylmethylcellulose and sodium CMC are used to prepare the tablets. The pharmaceutical composition of this example is given in Table 2.

TABLE 2

| Ingredients | Quantity in mg per tablet |
|---|---|
| Metformin hydrochloride | 500 |
| Microcrystalline cellulose (MCC) | 59 |
| Povidone (PVP K-30) | 15 |
| Sodium bicarbonate | 70 |
| Hydroxypropylmethylcellulose K-100 M | 130 |
| Sodium carboxymethylcellulose | 15 |
| (Cekol ® 10000A) | |
| Sodium Starch Glycolate | 4 |
| Aerosil ® 200 | 5 |
| Magnesium stearate | 2 |

i. Binder solution was prepared by dissolving povidone in isopropyl alcohol.
ii. Metformin hydrochloride that was sieved through 80-mesh sieve, microcrystalline cellulose, and hydropropylmethylcellulose were mixed properly and granulated with the binder solution of step (i).
iii. The wet mass obtained in step (ii) was passed through 8-mesh sieve and dried in a drier.
iv. The dried granules obtained in step (iii) were resized through 18-mesh sieve and blended with sodium bicarbonate, sodium carboxymethylcellulose, sodium starch glycolate, Aerosil® and magnesium stearate.
v. The resultant lubricated blend obtained in step (iv) is compressed to a tablet so as to provide about 500 mg of metformin hydrochloride.

### Example 3

This example illustrates the present invention in the form of controlled release tablets of metformin hydrochloride wherein a combination of a hydrophilic polymers Hydroxypropylmethylcellulose, hydroxyethyl cellulose and a third hydrophilic polymer (sodium carboxymethylcellulose) is used to prepare the tablets. The pharmaceutical composition of this example is given in Table 3.

TABLE 3

| Ingredients | Quantity in mg per tablet |
|---|---|
| Metformin hydrochloride | 500 |
| Povidone (PVP K-30) | 15 |
| Sodium bicarbonate | 70 |
| Hydroxypropylmethylcellulose K-100 M | 160 |
| Hydroxyethylcellulose (HHX Pharm) | 29 |
| Sodium carboxymethylcellulose | 15 |
| (Cekol ® 10000A) | |
| Sodium Starch Glycolate | 4 |
| Aerosil ® 200 | 5 |
| Magnesium stearate | 2 |

i. Binder solution was prepared by dissolving povidone in isopropyl alcohol.
ii. Metformin hydrochloride that was sieved through 80-mesh sieve and hydropropylmethylcellulose were mixed properly and granulated with the binder solution of step (i).
iii. The wet mass obtained in step (ii) was passed through 8-mesh sieve and dried in a drier.
iv. The dried granules obtained in step (iii) were resized through 18-mesh sieve and blended with sodium bicarbonate, hydroxyethylcellulose, sodium carboxymethylcellulose, sodium starch glycolate, Aerosil® and magnesium stearate.
v. The resultant lubricated blend obtained in step (iv) is compressed to a tablet so as to provide about 500 mg of metformin hydrochloride.

### Example 4

This example illustrates the present invention in the form of controlled release tablets of metformin hydrochloride wherein a combination of a hydrophilic polymers hydroxypropylmethylcellulose and sodium carboxymethylcellulose and guar gum is used to prepare the tablets. The pharmaceutical composition of this example is given in Table 4.

TABLE 4

| Ingredients | Quantity in mg per tablet |
|---|---|
| Metformin hydrochloride | 500 |
| Povidone (PVP K-30) | 15 |
| Sodium bicarbonate | 70 |
| Hydroxypropylmethylcellulose K-100 M | 94.5 |
| Guar gum | 94.5 |
| Sodium carboxymethylcellulose | 15 |
| (Cekol ® 10000A) | |
| Sodium Starch Glycolate | 4 |
| Aerosil ® 200 | 5 |
| Magnesium stearate | 2 |

i. Binder solution was prepared by dissolving povidone in isopropyl alcohol.
ii. Metformin hydrochloride that was sieved through 80-mesh sieve, guar gum and hydroxypropylmethylcellulose were mixed properly and granulated with the binder solution of step (i).
iii. The wet mass obtained in step (ii) was passed through 8-mesh sieve and dried in a drier.
iv. The dried granules obtained in step (iii) were resized through 18-mesh sieve and blended with sodium bicarbonate, sodium carboxymethylcellulose, sodium starch glycolate, Aerosil® and magnesium stearate.
v. The resultant lubricated blend obtained in step (iv) is compressed to a tablet so as to provide about 500 mg of metformin hydrochloride.

### Example 5

This example illustrates the present invention in the form of controlled release tablets of metformin hydrochloride wherein a combination of a hydrophobic polymer polyethyleneoxide and a hydrophilic polymer sodium carboxymethylcellulose are used to prepare the tablets. The pharmaceutical composition of this example is given in Table 5.

TABLE 5

| Ingredients | Quantity in mg per tablet |
|---|---|
| Metformin hydrochloride | 500 |
| Povidone (PVP K-30) | 15 |
| Sodium bicarbonate | 70 |
| Polyethyleneoxide (PEO 18 NF) | 189 |
| Sodium carboxymethylcellulose | 15 |
| (Cekol ® 10000A) | |
| Sodium Starch Glycolate | 4 |
| Aerosil ® 200 | 5 |
| Magnesium stearate | 2 |

i. Binder solution was prepared by dissolving povidone in isopropyl alcohol.
ii. Metformin hydrochloride that was sieved through 80-mesh sieve and polyethyleneoxide were mixed properly and granulated with the binder solution of step (i).
iii. The wet mass obtained in step (ii) was passed through 8-mesh sieve and dried in a drier.
iv. The dried granules obtained in step (iii) were resized through 18-mesh sieve and blended with sodium bicarbonate, sodium carboxymethylcellulose, sodium starch glycolate, Aerosil® and magnesium stearate.
v. The resultant lubricated blend obtained in step (iv) is compressed to a tablet so as to provide about 500 mg of metformin hydrochloride.

### Example 6

This example illustrates the present invention in the form of controlled release tablets of metformin hydrochloride wherein a combination of a hydrophobic polymer Eudragit® RS 100 and a hydrophilic polymer sodium carboxymethylcellulose are used to prepare the tablets. The pharmaceutical composition of this example is given in Table 6.

TABLE 6

| Ingredients | Quantity in mg per tablet |
|---|---|
| Metformin hydrochloride | 500 |
| Povidone (PVP K-30) | 15 |
| Sodium bicarbonate | 70 |
| Eudragit ® RS 100 | 80 |
| Microcrystalline cellulose | 106 |
| Sodium CMC | 15 |
| (Cekol ® 10000A) | |
| Sodium Starch Glycolate | 6 |
| Aerosil ® 200 | 5 |
| Magnesium stearate | 3 |

i. Binder solution was prepared by dissolving povidone in isopropyl alcohol.
ii. Metformin hydrochloride that was sieved through 80 mesh sieve and Eudragit® RS 100 were mixed properly and granulated with the binder solution of step (i).
iii. The wet mass obtained in step (ii) was passed through 8-mesh sieve and dried in a drier.
iv. The dried granules obtained in step (iii) were resized through 18-mesh sieve and blended with sodium bicarbonate, sodium carboxymethylcellulose, sodium starch glycolate, Aerosil® and magnesium stearate.
v. The resultant lubricated blend obtained in step (iv) is compressed to a tablet so as to provide about 500 mg of metformin hydrochloride.

### Example 7

This example illustrates the present invention in the form of controlled release tablets of metformin hydrochloride wherein a combination of a hydrophilic polymers carbopol 714 and sodium carboxymethylcellulose are used to prepare the tablets. The pharmaceutical composition of this example is given in Table 7.

TABLE 7

| Ingredients | Quantity in mg per tablet |
|---|---|
| Metformin hydrochloride | 500 |
| Povidone (PVP K-30) | 15 |
| Sodium bicarbonate | 70 |
| Carbopol 714 | 150 |
| Microcrystalline cellulose | 39 |
| Sodium CMC | 15 |
| (Cekol ® 10000A) | |
| Sodium Starch Glycolate | 4 |
| Aerosil ® 200 | 5 |
| Magnesium stearate | 2 |

i. Binder solution was prepared by dissolving povidone in isopropyl alcohol.
ii. Metformin hydrochloride that was sieved through 80 mesh sieve, microcrystalline cellulose and carbopol 714 were mixed properly and granulated with the binder solution of step (i).
iii. The wet mass obtained in step (ii) was passed through 8-mesh sieve and dried in a drier.
iv. The dried granules obtained in step (iii) were resized through 18-mesh sieve and blended with sodium bicarbonate, sodium carboxymethylcellulose, sodium starch glycolate, Aerosil® and magnesium stearate.
v. The resultant lubricated blend obtained in step (iv) is compressed to a tablet so as to provide about 500 mg of metformin hydrochloride.

### Example 8

This example illustrates the present invention in the form of controlled release tablets of metformin hydrochloride wherein a combination of a hydrophobic polymer Compritol® 888ATO and hydrophilic polymers Hydroxyethylcellulose and sodium carboxymethylcellulose are used to prepare the tablets. The pharmaceutical composition of this example is given in Table 8.

TABLE 8

| Ingredients | Quantity in mg per tablet |
|---|---|
| Metformin hydrochloride | 500 |
| Povidone (PVP K-30) | 15 |
| Sodium bicarbonate | 80 |
| Compritol ® 888ATO | 120 |
| hydroxyethylcellulose (HHX Pharm) | 30 |
| Microcrystalline cellulose | 21 |
| Sodium CMC | 15 |
| Sodium Starch Glycolate | 4 |
| Citric Acid | 8 |
| Aerosil ® 200 | 5 |
| Magnesium stearate | 2 |

i. Binder solution was prepared by dissolving povidone in isopropyl salcohol.
ii. Metformin hydrochloride that was sieved through 80 mesh sieve, microcrystalline cellulose and Compritol® 888ATO were mixed properly and granulated with the binder solution of step (i).
iii. The wet mass obtained in step (ii) was passed through 8-mesh sieve and dried in a drier.
iv. The dried granules obtained in step (iii) were resized through 18-mesh sieve and blended with sodium bicarbonate, Hydroxyethylcellulose, sodium carboxymethylcellulose, sodium starch glycolate, citric acid, Aerosil® and magnesium stearate.
v. The resultant lubricated blend obtained in step (iv) is compressed to a tablet so as to provide about 500 mg of metformin hydrochloride.

### Dissolution “In Vitro”

The tablets were characterised for drug release in 900 ml of 0.1 N hydrochloric acid. The USP apparatus Type II with paddle speed at 50 rpm was used at 37° C. The samples of the media were periodically withdrawn and analysed for drug content. The results are shown in Table 9.

TABLE 9

| | |
|---|---|
| Time | % of Metformin released |

| (Hours) | Ex. 3 | Ex. 4 |
|---|---|---|
| 2 | 35.6 | 41.4 |
| 4 | 54.6 | 62.4 |
| 6 | 67.0 | 77.3 |
| 8 | 81.2 | 88.0 |
| 12 | NLT 95 | NLT 95 |

NLT = Not less than

The tablets were characterized for time required to float on the surface as well the total floating period by immersing in 0.1 N hydrochloric acid. The results are shown in Table 10.

TABLE 10

| Example No. | Time to float on surface | Floating period | |
|---|---|---|---|
| 3 | 1 minute 30 seconds | 10-12 | hours |
| 4 | 1 minute 30 seconds | 8 | hours |

### Pharmacokinetic Studies:

The composition of example 3 (herein after defined as test composition) was the object of a pharmacokinetic study in comparison with a metformin extended release composition (herein after defined as standard composition) already on the market. 12 healthy volunteers were randomized to receive 500 mg of the two products (either test composition or standard composition). Each drug administration was separated by a washout period of seven days.

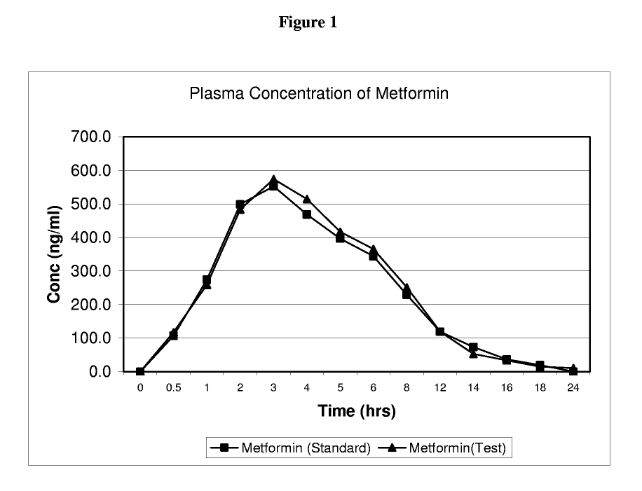

Blood samples (5 ml) were obtained from subjects at 0 (pre dose), 0.5, 1, 2, 3, 4, 5, 6, 8, 12, 14, 16, 18 and 24 hour(s). Plasma concentrations of metformin were determined using a validated HPLC method. Mean plasma concetration time profiles are shown in FIG. 1 and mean values of pharmakokinetic parameters of metformin obtained from this study are presented in Table 11.

TABLE 11

| Composition | Cₘₐₓ | AUC₍₀₋₂₄₎ | AUC_{(0-□)} |
|---|---|---|---|
| Test | 566.34 | 3833.76 | 4399.23 |
| Standard | 569.77 | 3620.93 | 4229.39 |
| Ratio: Test/Standard | 0.994 | 1.059 | 1.04 |

1. An extended release pharmaceutical composition in the form of a tablet, comprising metformin or its pharmaceutically acceptable salt; a gas generating agent; a hydrophilic or hydrophobic polymer as release retardant; a disintegrant; one more hydrophilic polymer to provide system stability; additionally a hydrophilic polymer or a gum as release modifier and optionally other pharmaceutical excipients.

2. The pharmaceutical composition of claim 1, wherein the composition exhibits the following dissolution profile when tested in a USP type II apparatus at 50 rpm in 900 ml of 0.1 N HCI at 37° C.:
10-45% of the metformin or salt thereof is released after 2 hours;
40-65% of the metformin or salt thereof is released after 4 hours;
60-80% of the metformin or salt thereof is released after 6 hours;
75-95% of the metformin or salt thereof is released after 8 hours;
not less than 95% of the metformin or salt thereof is released after 12 hours;

3. The pharmaceutical composition of claim 2, wherein the composition provides a mean time to maximum plasma concentration (Tₘₐₓ) of metformin from 2.0 to 4.0 hours after the administration of dose.

4. The pharmaceutical composition of claim 2, wherein the composition provides a mean maximum plasma concentration (Cₘₐₓ) of metformin from about 450 ng/ml to about 650 ng/ml, based on administration of a 500 mg once-a-day dose of metfromin.

5. The pharmaceutical composition of claim 1 wherein the gas generating agent is selected from magnesium carbonate, sodium bicarbonate or potassium bicarbonate.

6. The composition as claimed in claim 5, wherein the gas generating agent is sodium bicarbonate.

7. The pharmaceutical composition of claim 1, wherein the hydrophilic or hydrophobic polymer as release retardant is selected from hydroxylethylcellulose, polyvinylpyrrolidone in combination with poly(vinyl alcohol), hydroxypropylcellulose, hydroxymethylcellulose, hydroxypropylmethylcellulose, gelatin, polyacrylic acid (carbopol), polyethyleneoxide, Eudragit®, Compritol®, polypropylene oxide, polyethylene, polypropylene, polycarbonate, polystyrene, polysulfone, polyphenylene oxide, polytetramethylene ether and combinations thereof.

8. The pharmaceutical composition as claimed in claim 7, wherein the hydrophilic or hydrophobic polymer as a release retardant polymer is selected from hydroxypropylmethylcellulose, polyacrylic acid (carbopol), polyethyleneoxide, hydroxyethylcellulose, Eudragit®, Compritol® and combinations thereof.

9. The pharmaceutical composition of claim 1, wherein the disintegrant is selected from crospovidone, croscarmellose sodium, sodium starch glycolate, low-substituted hydroxypropyl cellulose and or combinations thereof.

10. The pharmaceutical composition as claimed in claim 9, wherein the disintegrant is sodium starch glycolate.

11. The pharmaceutical composition of claim 1 wherein the hydrophilic polymer to provide system stability is selected from sodium carboxymethylcellulose, hydroxyethylcellulose, hydroxypropylmethylcellulose and mixtures thereof.

12. The pharmaceutical composition as claimed in claim 11, wherein the hydrophilic polymer to provide system stability is selected from sodium carboxymethylcellulose and hydroxypropylmethylcellulose.

13. The pharmaceutical composition of claim 1, wherein the additional hydrophilic polymer or a gum as a release modifier is selected from sodium carboxymethylcellulose, guar gum, gum arabic, locust bean gum, xanthan gum and combinations thereof.

14. The pharmaceutical composition as claimed in claim 13, wherein the hydrophilic polymer or gum as release modifier is selected from sodium carboxymethylcellulose and guar gum.

15. The pharmaceutical composition as claimed in claim 1 comprising about 55 to about 70% w/w metformin or a pharmaceutically acceptable salt thereof; about 5 to about 15% w/w gas generating agent; about 5 to about 50% w/w hydrophilic and/or hydrophobic polymer or gum.

16. The pharmaceutical composition as claimed in claim 15 comprising about 60 to about 65% w/w metformin or a pharmaceutically acceptable salt thereof; about 7 to about 10% w/w gas generating agent; about 7.5 to about 35% w/w hydrophilic and/or hydrophobic polymer or gum.

17. The pharmaceutical composition of claim 1, further comprising pharmaceutical excipients selected from a filler, a binder, a glidant and a lubricant or mixtures thereof.

18. A process for preparing the pharmaceutical composition of claim 1 comprising the steps of:
(i) dissolving the binder in isopropyl alcohol followed by granulating metformin or its pharmaceutically acceptable salt and the hydrophilic and/or hydrophobic polymer and the additional hydrophilic polymer or gum
(ii) passing the resultant wet mass obtained in step (i) through a sieve and drying the resultant wet granules in a drier;
(iii) resizing the resultant dried granules obtained in step (ii) above and further mixing the same with a gas generating agent and other excipients including lubricant, glidant, binder and/or filler;
(iv) compressing the resultant lubricated blend to a tablet.

19. The pharmaceutical composition as claimed in claim 1, wherein the composition when immersed in 0.1 N hydrochloric acid, starts to float on the surface within about 0.5 minute to about 5 minutes and continues to float for a period between about 6 hours to about 14 hours.
